(19) United States
(12) Patent Application Publication
(10) Pub. No.: US 2023/0118114 A1
(43) Pub. Date: Apr. 20, 2023

(54) BIOERODIBLE LIFE SUPPORT HYDROGELS FOR THE DELIVERY OF VIABLE MITOCHONDRIA

(71) Applicant: University of Kentucky Research Foundation, Lexington, KY (US)

(72) Inventors: Thomas D. Dziubla, Lexington, KY (US); Md. Arif Khan, Lexington, KY (US); Alexander G. Rabchevsky, Nicholasville, KY (US); Samirkumar P. Patel, Lexington, KY (US); Jason DeRouchey, Lexington, KY (US); Patrick G. Sullivan, Nicholasville, KY (US)

(21) Appl. No.: 17/959,809

(22) Filed: Oct. 4, 2022

Related U.S. Application Data
(60) Provisional application No. 63/251,770, filed on Oct. 4, 2021.

(51) Int. Cl. A61K 35/34; A61K 9/06; A61P 25/00; A61K 47/38; A61K 47/36; A61K 31/16; A61K 31/225; A61K 9/00

(52) U.S. Cl. CPC A61K 35/34; A61K 9/06; A61K 9/0085; A61K 31/16; A61K 31/225; A61K 47/36; A61K 47/38; A61P 25/00

(57) ABSTRACT

This disclosure relates to hydrogel compositions with isolated mitochondria suspended therein. The compositions are for providing protection to injured tissue, as well as tissue proximal to a site of injury. The hydrogel provides structural support while the mitochondria are able to prevent or slow cellular death. In some aspects, the compositions include N-acetylcysteine amide and/or acetyl-L-carnitine.

### CROSS-RELATED APPLICATIONS

This application claims priority to U.S. Provisional Application 63/251,770, filed Oct. 4, 2021, the content of which is hereby incorporated by reference in its entirety.

### FIELD OF THE INVENTION

This disclosure relates to a hydrogel compositions and methods of the administration of the same to provide viable mitochondria to a site of injury and/or insult in a subject.

### GOVERNMENT INTEREST

This disclosure was supported by grants from the National Institute for Health (5R01NS119337-02) and the U.S. Department of Defense, Army Medical Research and Material Command (W81XWH2010347). The Government has certain rights to the invention.

### BACKGROUND

Following an injurious event or an insult to tissue, cells therein and proximal thereto lose significant numbers of mitochondria and, as a result, energy generation capacity declines, which can result in cell death and expansion of the injury site. To counteract the drop in energy generation, replenishing mitochondria at and/or around the site of injury or insult seems a straightforward solution. However, providing a system or method to provide healthy mitochondria remains problematic. Mitochondria transplantation (MitoTxp) by the delivery of exogenous healthy mitochondria to injured cells is technically challenging due to complicating factors such as the short time window mitochondria remain viable outside of their cytosolic environment, possible injection-induced secondary injury from delivering mitochondria, and mitochondria aggregate formation after delivery. Thus, there is a need for systems and methods that provide intact and healthy mitochondria to a site of injury or insult to counteract or prevent endogenous mitochondria depletion.

### BRIEF DESCRIPTION OF THE DRAWINGS

FIG. 1A shows behavioral (BBB) scores and tissue sparing for injured rats treated with acetyl-L-carnitine (ALC) vs. vehicle. *p<0.05 vs. vehicle (Patel et al., 2012).

FIG. 1B shows BBB scores and tissue sparing for injured rats treated with N-acetylcysteine amide (NACA) vs. vehicle, *p<0.05 vehicle vs. 150 mg/kg NACA and #p<0.05 vehicle vs. 300 mg/kg (Patel et al., 2014) Symbols represent means ± SEM..

FIG. 2 shows effects of Mito Txp (100 ug) or ALC (300 mg/kg, i.p.), alone or in combination, on mitochondrial respiration in terms of Oxygen Consumption Rate (OCR). Bars are Mean ± SD, n=5-6/group. *p<0.05 compared with sham; # p<0.05 versus vehicle.

FIGS. 3 shows representative images of cell type-specific MitoTracker Red CMX Ros-labeled mitochondria (MTR Mito) co-localization following intraspinal or subdural MitoTxp. Spinal cord cross sections were stained with specific cell type markers. Images in left panels represent Intraspinal and right panels represent subdural MitoTxp. Top panel images show transplanted MTR Mito in parenchyma via intraspinal (FIG. 3A) and subdural (FIG. 3F) routes. Pictures at 10 x (FIGS. 3B, 3D, 3G, 3I) and Z-stacked images (100 x FIGS. 3C, 3E, 3H, 3J) of boxed regions show co-localization (arrowheads) of MTR Mito proximal to neurons (NeuN: FIGS. 3B, 3C, 3G, 3H) and within microglia/brain macrophages (OX-42: FIGS. 3D, 3E, 3I, 3J). Intraspinal injections were associated with neuronal loss (FIG. 3B) compared to subdural routes (FIG. 3G). Scale bars for 10 x=100 µm, 100 x=10 µm

FIGS. 4 shows co-localization of MTR Mito following subdural delivery. (FIG. 4A) Regions of MTR Mito dispersion. Left panels (FIGS. 4B-4J) represent lower magnification of Z-stacked images of boxed regions in right panels (FIGS. 4C-4K). MTR Mito co-localized with inner mitochondria membrane (FIG. 4B, FIG. 4C; COXIV), around neurons (FIG. 4D, FIG. 4E; NeuN), and within endothelial cells (FIG. 4F- FIG. 4G; RECA), pericytes (FIG. 4H, FIG. 4I; PDGF-B) and brain macrophages (FIG. 4J, FIG. 4K; Ox42). DAPI (4′,6-diamidino-2-phenylindole), cell nuclei. Scale bars =100 µm (FIGS. 4A-4J), 10 µm (C-K).

FIG. 5 shows optical density of (a) MC and (b) HAMC hydrogels with temperature. Various compositions of HAMC have been prepared. UV-vis absorbance was measured for the samples while ramping the temperature from 4° C. to 70° C. in 5° C. intervals.

FIG. 6 shows differential scanning calorimetry of MC, HA, and the composite of both. Both heating (a) and cooling (b) cycles show clear phase separation of MC from water at around 40C. HA sample in both heating (c) and cooling (d) cycles does show much change. HAMC show a clear broad inclination and a small flat behavior around 60° C. showing phase separation. Samples’ weight 8.4 mg with 5% w/w polymer concentration.

FIG. 7 HA and MC gel dissolution. Both polymers are labelled with dyes (HA-fluorescein, MC- EDANS). The results of HA (b) show a gradual release reaching the maximum after almost 24 hrs. The MC release (d) is faster reaching the maximum within about 2 hrs.

FIG. 8 shows fluorescence intensity counts for FHA-EDMC gel degradation using micro-plate reader. Excitation 498/9, emission 520/9, gain 60, data points 10. a) shows concentrations of samples as collected; b) shows cumulative FHA released (mg).

FIG. 9 shows fluorescence intensity counts for FHA-EDMC gel degradation. Excitation 355/9, emission 455/9, gain 90, data points 10. a) shows concentrations of samples as collected; b) shows cumulative FHA released (mg).

FIG. 10 shows the release profiles for polystyrene beads with varying sizes from the indicated hydrogels.

FIG. 11 shows the profile of mitochondrial release from the indicated gels.

### DESCRIPTION

The present disclosure concerns compositions and methods to provide transplanted delivery of healthy mitochondria at and/or around the site of an injury or surgical incision or other tissue insult in a controlled manner from biodegradable polymeric hydrogels. As set forth herein, the hydrogels after transplantation start to or effectively achieve ‘homeostasis’ as indicated by both improved cellular bioenergetics accompanied by reduced levels of reactive oxygen species (ROS). The present compositions and the methods of the present disclosure can be applied to any type of injury, including spinal cord injury (SCI), cardiac ischemic events and pulmonary ischemic injuries. The success provided by the compositions and methods of the present disclosure is brought upon by the inherent rapid delivery and uptake of mitochondria in tissue.

### Compositions

In some aspects, the present disclosure concerns hydrogel compositions that include mitochondria that can be applied at the site of an injury or insult to reduce energy depletion and impede the negative damaging cascades that can result in cell death. In some aspects, the present disclosure concerns a hydrogel that can be delivered to the site of injury, not into the parenchyma. Such an approach translates the first step to transplanting mitochondria from either exogenous or autologous sources in the acute stages after injury in the clinic to foster rapid reduction in oxidative stress to prevent secondary injury cascades into the penumbral zone.

In some aspects, the present disclosure concerns compositions for MitoTxp, the compositions being of a hydrogel with mitochondria suspended therein. The compositions can be applied to a site of injury or insult to provide an enhanced MitoTxp that demonstrates improved mitochondrial viability and cellular uptake thereof. In some aspects, the hydrogel is a cross-linked hydrophilic polymer that can swell in an aqueous environment and/or retain water therein. In some aspects, a hydrogel may include a homopolymer hydrogel, a copolymer hydrogel, or a multipolymer interpenetrating polymeric hydrogel. In other aspects, the hydrogel may be an amorphous or non-crystalline hydrogel, a semicrystalline hydrogel, or a crystalline hydrogel. In further aspects, the hydrogel may form by covalent bonds, hydrogen bonds, polymer chain entanglement, ionic interaction(s), hydrophobic interaction(s), or combinations thereof. In other aspects, the hydrogel may be a nonionic hydrogel, an ionic hydrogel, an ampholytic hydrogel or a zwitterionic hydrogel.

In some aspects, the hydrogel may form and/or be sensitive to surrounding conditions and/or environmental conditions, such that a change therein can cause the hydrogel to form and/or swell or to disintegrate and/or deswell. In some aspects, the hydrogel may be sensitive to changes or modifications to the environmental temperature, electric field, magnetic field, light, pressure, sound, pH, ionic strength, and/or sound, as well as to surrounding molecules and/or solvents. In some aspects, a temperature responsive hydrogel wherein gelation occurs above room temperature (RT), such as at or near body temperature, allows for the hydrogel of the present disclosure to polymerize when in situ within a subject receiving MitoTxp. It will be appreciated, however, that the present disclosure need not be limited to thermo-gelling hydrogels.

In some aspects, the compositions of the present disclosure concern thermo-hydrogels or thermo-gelling hydrogels of hyaluronic acid (HA) and methylcellulose (MC). Both HA and MC are recognized as biocompatible in human subjects and can be easily tuned and possess-thermo gelling properties. Particularly, HA and MC (HAMC) hydrogels at cooler temperatures such as 4° C. can present as a viscous liquid and/or sol phase, allowing for application of the composition to the site of an injury or insult while avoid backflow and providing enhanced gelation required to prevent mitochondrial aggregates from forming. Further, once in situ at the site of injury and/or region/space surrounding the injury, the thermo-hydrogel can shift to a gel phase or harden in response to the rise in temperature provided by the subject’s body temperature. It will be appreciated that other temperature sensitive polymers can be included or substituted therein, such as N-isopropylacrylamide (PNiPAAm), chitosan, pluronic or poloxamers of poly(ethylene oxide)-b-poly(propylene oxide)-b-poly(ethylene oxide), polyethylene glycol (PEG), PEG acrylate, PEG thiol-ene, acrylic acid and esters thereof, methyl acrylate, butyl acrylate, ethyl acrylate, 2-ethylhexyl acrylate, gelatin, laminin, mucin, sericin, fibrin, alginate, heparin, and/or polyvinyl alcohol.

In some aspects, the thermo-gelling hydrogels of the compositions including a mixture of HA and MC. In some aspects, the thermo-gelling hydrogel may be of from about 0.1 to about 20% by weight of the composition, including about 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, and 19% by weight of the composition. In further aspects, the composition may include a thermo-gelling hydrogel that includes both HA and MC, wherein the ratio of HA to MC is of from about 1:20 to about 20:1, including 1:19, 1:8, 1:17, 1:16, 1:15, 1:14, 1:13, 1:12, 1:11, 1:10, 1:9, 1:8, 1:7, 1:6, 1:5, 1:3, 1:2, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, and 19:1 of HA to MC.

In some aspects, one more supportive agent(s) may be included within the hydrogel to provide for mitochondrial health and/or improve tissue healing may be suspended in the hydrogel. In some aspects, the hydrogel may include a nano and/or microparticle that provides controlled release of one or more supportive agents therein that can further assist with mitochondrial health and/or improved tissue healing at the site of action. By providing a nanoparticle and/or microparticle within the hydrogel, the release of the one or more supportive agents can be further protected, controlled and/or sustained. For example, in some aspects, it may be desirable to include a therapeutic agent within the hydrogel to aid in the healing process. Encapsulation of such within a nano and/or micro particle within the hydrogel protects from unwanted degradation and/or oxidation thereof, as well as providing a platform for sustained and/or controlled release during the time course of healing.

Suitable materials for nano and/or microparticles are generally understood. Such can include lipids, biodegradable polymers, biodurable polymers, saccharides, carbohydrates and similar. Examples include poly(ethylene glycol) (PEG), poly(lactic acid) (PLA), poly glycolic acid (PGA), poly(lactic-co-glycolic acid) (PLGA), poly(caprolactone) (PCL), poly (β-amino ester) (PβAE), chitosan, starch, cholesterol, distearoyl phosphocholine, 1,2-dioleoyl-3-trimethylammoniumpropane (DOTAP) and/or dioleoylphosphatidylethanolamine (DOPE) and/or lipofectamine and/or dioleoylphosphatidylcholine (DOPC) and/or phosphatidylethanolamine (dioleoyl PE) and/or 3β-[N-(N′,N′-dimethylaminoethane)-carbamoyl]-cholesterol (DC-Chol) In some aspects, the nano and/or microparticle may be of a poly (β-amino ester) (PβAE) hydrogel. (See, e.g., U.S. Pat. 8,642,087 and 9,433,638).

In further aspects, the compositions of the present disclosure may include at least one mitochondrial protecting agent. In some aspects, the mitochondrial protecting agent may include compounds such as ALC and NACA. In some aspects, the composition may include ALC at a weight percentage of from about 0.1 to about 20% by weight of the thermo-gelling hydrogel, including about 0.2, 0.3, 0.4, 0.5, 0.6, 0.7, 0.8, 0.9, 1, 2, 3, 4, 5, 6, 7,, 8, 9, 10, 11, 12, 13, 14, 15, 16, 17, 18, and 19% by weight of the composition of the thermo-gelling hydrogel. In further aspects, the composition of the thermo-gelling hydrogel may include NACA at a weight percentage of from about 0.1 to about 20% by weight of the thermo-gelling hydrogel, including about 0.2, 0.3, 0.4, 0.5, 0.6, 0.7, 0.8, 0.9, 1, 2, 3, 4, 5, 6, 7,, 8, 9, 10, 11, 12, 13, 14, 15, 16, 17, 18, and 19% by weight of the composition of the thermo-gelling hydrogel. In some aspects, the thermo-gelling hydrogel composition may include both ALC and NACA, both being present at about 0.1 to about 20% by weight of the composition.

In some aspects of the present disclosure, the composition may include the mitochondrial protecting agent ALC or derivatives thereof. ALC and its derivatives can provide a source of energy or biofuel to assist in the production of adenosine triphosphate (ATP). For example, in animals with an induced spinal cord injury (SCI), once daily treatment ALC maintains mitochondrial respiration when administered acutely after contusion SCI, and continued daily treatment with ALC for three weeks significantly improves long term hind limb locomotor function and tissue sparing. ALC is an acetylated derivative of L-carnitine (LC), which is biosynthesized from the amino acids lysine and methionine and is known to be involved in numerous physiological reactions, including oxidative phosphorylation, fatty acid oxidation and aerobic sugar metabolism. ALC supplementation may provide neuroprotective effects in various age-related neurodegenerative disorders and is used to treatment of a variety of chronic neurological diseases. One of the potential mechanisms by which ALC exerts its protective effects is by increasing the uptake of acetyl CoA into mitochondria, where it enters the citric acid cycle and forms NADH, which is oxidized in the mitochondrial electron transport chain and donates electrons for ATP synthesis.

In some aspects of the present disclosure, the composition may include the mitochondrial protecting agent NACA or derivatives thereof. NACA or derivatives thereof may maintain acute mitochondrial bioenergetics and normalizes glutathione (GSH) levels. For example, continuous delivery of NACA for one-week post-SCI results in significant tissue sparing and improved recovery of hindlimb function. NACA is a modified form of its parent compound N-acetylcysteine (NAC) that is a precursor of the most abundant endogenous antioxidant, GSH. NAC is an FDA-approved, thiol-containing compound which acts as an acetylated cysteine precursor of GSH. GSH is synthesized by most cells within the body and is largely responsible for maintaining a balanced redox state. Levels of mitochondrial GSH are associated with the degree of tissue damage after insults or injury. However, it is well documented that NAC has restricted bioavailability, due to its low lipid solubility combined with negatively charged carboxyl groups at physiological pH, which limits its ability to cross cell membranes. To combat this problem, the amide derivative of NAC (‘NACA’) was designed to be more lipophilic and therefore cell-permeating, due to neutralization of the carboxylic group. This compound readily crosses the brain-blood barrier (BBB) and penetrates cellular and mitochondrial membranes to replenish intracellular GSH levels, scavenge free radicals and protect against oxidative stress.

In some aspects, the hydrogel compositions include mitochondria. In further aspects, a thermo-gelling hydrogel composition may include mitochondria suspended therein. The mitochondria may be derived from any cell or tissue type. In some aspects, the mitochondria may be homologous to the subject receiving the compositions. In other aspects, the mitochondria may be heterologous. In certain aspects, such as for the purposes of potential immunoreactivity, the mitochondria should be derived from the same species as the subject to receive the mitochondria. In further aspects, the mitochondria are prepared to be sufficiently pure to avoid potential immunoreactivity from the subject receiving the composition. The mitochondria can be prepared and/or obtained by any technique known in the art, including low speed centrifugation, high speed centrifugation, differential centrifugation, density gradient centrifugation and/or affinity purification(see, e.g., Liao et al., Methods Cell Biol. 155:3-31, 2020). In some aspects, the mitochondria are provided to the thermo-gelling hydrogel and mixed therein. It will be apparent to those in the art that the mitochondria be provided to the thermo-gelling hydrogel prior to any gelling event. Such may be achieved by adding the mitochondria at a sufficiently low temperature that the thermo-gelling hydrogel is in a liquid state, or into an aqueous solution prior to the addition of HA and/or MC to generate the hydrogel. The amount of mitochondria included may be dependent on the volume of the thermo-gelling hydrogel, as the formation of aggregates is possible with higher concentrations, yet the user will want to maximize the number of mitochondria delivered to the subject to increase the concentration of cellular uptake and provide the greater opportunity to reduce energy depletion and tissue damage at the site of injury or insult. In some aspects, the mitochondria may be of about 0.1 to about 30% by weight of the composition, including 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, and 29% by weight of the composition. In some aspects the mitochondria may be freshly prepared and added directly to the composition after harvesting or within 2 hours thereof, including 5, 10, 15, 20, 25, 30, 35, 40, 45, 50, 55, 60, 65, 70, 75, 80, 90, 95, 100, 105, 110, and 115 minutes thereof.

In some aspects, the hydrogel compositions may include additional components within the hydrogel, such as water, buffers, polymers, chelating agents, surfactants, emulsifiers, and albumin. It will be apparent that as the thermo-gelling hydrogel composition is to be applied to or is capable of being applied to an open wound, injury or insult on a subject, the thermo-gelling hydrogel should be sterile. As such, water utilized to prepare the hydrogel should of sufficient purity and/or sterility to ensure safety for the subject. In some other aspects, the hydrogel may include one or more buffers, such as phosphor-buffered saline (PBS), HEPES, Tris, or similar. In further aspects, the hydrogel may include one or more biocompatible polymers, such as poly(ethylene glycol) (PEG), poly(lactic acid) (PLA), poly glycolic acid (PGA), poly(lactic-co-glycolic acid) (PLGA), poly(caprolactone) (PCL), poly (N-isopropylacrylamide) (PNIPAAm), poly ((DL-lactic acid-co-glycolic acid)-g-ethylene glycol (PLGA-g-PEG), poly(caprolactone/ethylene glycol) copolymer (PCL-PEG), poly (caprolacton/lactide) copolymer (PCL-PLA), chitin, chitosan, alginate, collagen, gelatin, or combinations thereof.

In some aspects, the present disclosure concerns compositions of hydrogels with additional optional components suspended or dispersed therein. In some aspects, additional components may be dissolved within the hydrogel or provided as nanoparticles throughout the hydrogel. It will be apparent to those in the art that delivering or application of a hydrogel to a site of injury or insult provides an opportunity to include other optional components that may be of benefit to the subject receiving the treatment. Such additional optional components may include components that help with wound healing and/or prevent scarring and/or prevent infection. Such may include vitamins such as vitamin C, D, E, B6, B12, as well as amino acids or branched amino acids. Additional optional components may also include minerals, metals, and ions, such as iron, magnesium, calcium, sodium, potassium, silver, gold, manganese, phosphates, sulfates, chlorides. Additional optional components may also include co-factors for energy production, such as glucose or metabolites thereof, fructose, or other sugars, NADH, NADPH, isolated enzymes for glucose metabolism and/or the citric acid cycle and/or oxidative phosphorylation. Additional optional components may also include pharmaceutical compounds or therapeutics such as antiinflammatories, anti-fibrotics, steroids, anti-bacterials, anti-virals, anti-fungals. Additional optional components may also include isolated proteins to assist in wound healing such as albumin and/or collagens.

### Methods of Use

In some aspects, the present disclosure concerns the administration of the compositions set forth herein to a subject. In some aspects, the subject is a mammal, such as a human. It will however be appreciated that the protective effects provided by the compositions set forth herein are not limited to mammalian or even vertebrate animals only, but can aid any eukaryotic organism.

In some aspects, the present disclosure concerns administration or application of the compositions set forth herein to a site of injury and/or insult in a subject. Such can include incisions, lacerations, fractures, contusions, burns, lumpectomies, biopsies, surgeries, transplants, resections and amputations. As identified herein, the compositions of the present disclosure provide polymeric gels that provide to a site of injury mitochondria. The hydrogels further provide protection to the tissue both in the structure and support provided to the site of the injury, as well as in preventing access to infection by microbials.

Administration or application of the compositions can be through direct application to the site of injury, such as by pasting, pouring, painting, and injecting the composition to the site of injury and/or tissue surrounding the injury. In applications where the hydrogel is in the gel phase, application can be through placing a pre-formed gel at the site of injury or by sculpting or molding the hydrogel to a desired shape or configuration to occupy and fill the necessary space. In applications where the hydrogel is in a sol phase, the hydrogel may be poured or injected into the desired space. It will be appreciated that in instances of using a needle for application, care should be exercised to avoid unwanted shear of the mitochondria in the cannula of the needle.

The administration or application of the hydrogel compositions can be to any tissue. It will be appreciated that in some aspects, the hydrogel may possess degrees of tunability for hardness thereby allowing the compositions to be suitable for hard and soft tissues.

In further aspects, administration or application of the hydrogel compositions need not require open access to the site of injury. As identified herein, having the hydrogel in a sol phase allows for the composition to flow to a desired site, such as through the inner lumen of a tube or needle. Once in place, the hydrogel can transition to the gel phase. In some aspects, administration may be intrathecal, subdural, subdermal, intramuscular and similar.

In some aspects of the present disclosure, the methods of the present disclosure concern providing a hydrogel to a site of injury and/or tissue surrounding a site of injury. It will be appreciated that in some aspects, providing the hydrogel compositions to surround tissue can be protective and/or preventative against spreading necrosis. It will also be appreciated that the hydrogel compositions in their gel phase provide support to the site of injury and/or surrounding tissue.

In further aspects of the present disclosure, providing the hydrogel compositions to a site of injury provides a support that may degrade at a desired rate. In some aspects, the hydrogel composition degrades as the injured tissue heals, thereby allowing regenerated tissue to fill the space and avoid collapse or caving of other tissue.

In some aspects of the present disclosure, the application or administration of the hydrogel compositions provides viable mitochondria to a site of injury and/or tissue surround such. In other aspects, the hydrogel compositions provide mitochondria over a time course of healing. By suspending the mitochondria within the hydrogel, preservation of the mitochondria is achieved, while also controlling release into the tissue. Similarly, in aspects where supporting agents, NACA, ALC, therapeutics, nanoparticles, microparticles and combinations thereof are suspended within the hydrogel, their release and availability to the tissue is controlled. In additional aspects, the presence of nano and/or microparticles within the hydrogel composition provides a further option to control and/or sustain the release of any contents therein, such as therapeutics, NACA and/or ALC. The degradation of the nano and/or micro particle itself can control the release of contents therein, thereby providing a sustained and/or controlled release of the nano and/or microparticles into the site of injury and/or surrounding tissue and a sustained and/or controlled release of the contents therein.

### Examples

### Gel Preparation

Temperature sensitive HAMC hydrogels were acquired based on desired composition and added to a buffer of DI water and PBS and Mito isolation buffer. The mixture was vortexed for 10 sec and then centrifuged at 13000 rpm for 120 sec and then incubated on ice for 30 min. The vortex, centrifuge-chill cycle was repeated at least twice to ensure homogeneity. Gels were then left overnight inside an ice box on a shaker at 50 rpm.

Pure MC gels were compared with HAMC gels with thermogelation, optical densitry and differential scanning calorimetry. HAMC showed a clear broad inclination and a small flat behavior around 60° C. showing phase separation.

Fluorescent HA and MC gels were then prepared to assess hydrogel dissolution. Fluorescent HA (10 mL sodium hyaluronate in H₂O (50 mg, mixed with 5 mL DMSO, 0.5mL fluoresceinamine and 25 mL cyclohexyl isocyanide and 25 mL acetaldehyde, followed by 5 hr stirring in dark and adding few drop saturated NaCl) and fluorescent MC (200 mg MC in H₂O with 26.4 mg sodium periodate, stirred 24 hr, dialyzed, lyophilized and oxidized; oxidized MC mixed with 20 mg EDANS (dye) and 100 mg sodium cyanoborohydride, stirred 24 hr, dialyzed and lyophilized) were reviewed to observed release and dissolution. HA gels showed gradual release reaching aa maximum after almost 24 hrs. MC gels showed a faster release, reaching the m,aximum within about 2 hrs. These data show that the hydrogels are useful for retaining mitochondria for a period of time until they get dissolved unto surrounding body tissue(s).

### Animal Model

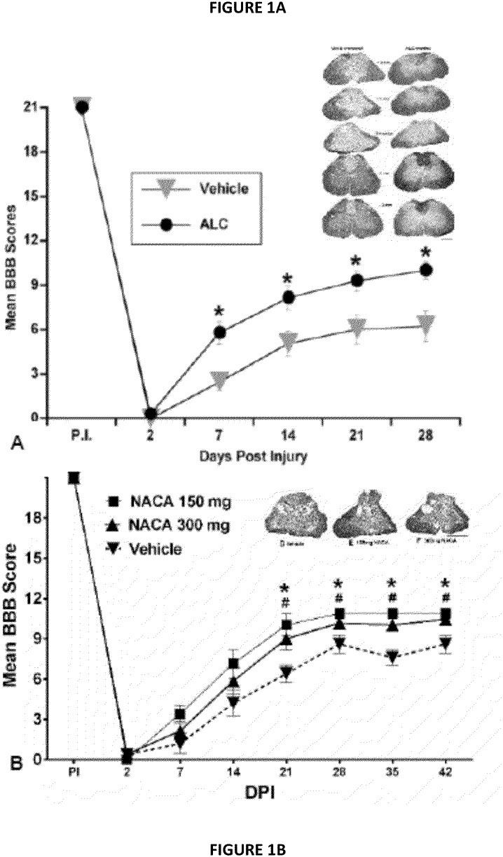

It has been identified that following a spinal cord injury (SCI), early protection of mitochondrial function with ALC is associated with increased neuroprotection and improved functional recovery at later stages. Briefly, after 3 weeks of daily ALC treatment, injured rats were able to step consistently with weight support on their hind limbs compared to vehicle-treated rats that showed only slight movements of hind limb joints (FIG. 1A). Such significant functional recovery was correlated with increased gray and white matter tissue sparing.

Studies also identified that continuous treatment with 150 and 300 mg/kg/dayNACA up to 7 days post-injury significantly increased long-term functional recovery and tissue sparing (FIG. 1B). However, since both ALC and NACA were only partially effective in improving hind limb locomotor function, it was identified that ALC and/or NACA, in combination with MitoTxp, is likely to provide additional protective effects at mitochondrial as well as cellular levels.

Since ALC acts as an alternative biofuel for mitochondria and NACA reduces oxidative stress, ALC and/or NACA likely not only improve function of mitochondria in injured spinal cords, but also increase survival and functionality of transplanted mitochondria, no matter what delivery means. These combinational approaches aim to improve mitochondrial bioenergetics and reduce oxidative stress to foster functional neuroprotection after contusion SCI.

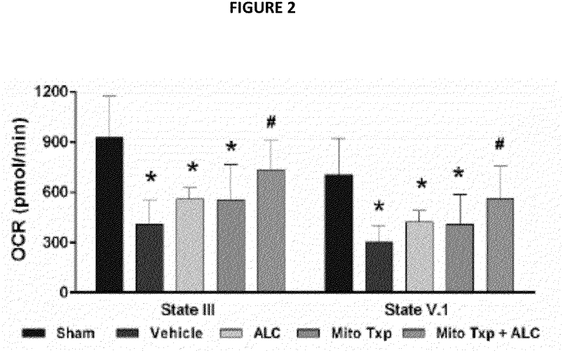

Recent studies have found that providing alternative biofuel in combination with MitoTxp results in significant maintenance of mitochondrial integrity compared to MitoTxp alone. Specifically, in combination with administration of ALC (300 mg/kg, i.p.) at 3 hours post-SCI, intraspinal MitoTxp significantly maintained mitochondrial bioenergetics compared to either alone (FIG. 2). Briefly, rats were contused at L1/L2 spinal level with 250 kDyn force using Infinite Horizon Impactor; Sham group received only T12 laminectomy. Designated injured rats were injected either intraspinally with healthy mitochondria (100 µg, MitoTxp) isolated from syngeneic rat soleus muscle within 3 hr and/or injected with ALC (300 mg/kg, i.p.) at 3 hours followed by a booster 6 hours later. Then, after 24 hours, mitochondria were isolated from spinal cord injury site and assessed for respiration (OCR). SCI significantly reduced States III & V.1 OCR vs. Shams, but MitoTxp + ALC together significantly maintained OCRs to Sham levels, whereas MitoTxp or ALC alone showed insignificant trends (FIG. 2). These data indicate synergistic effects of this novel combinational approach, as well as the potential for extending clinically relevant therapeutic time windows of combinational treatment (i.e., ALC at 3 hours and then MitoTxp at 24 hours).

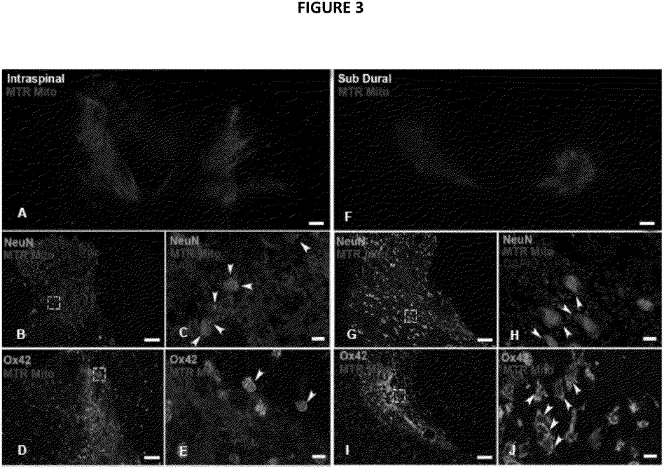

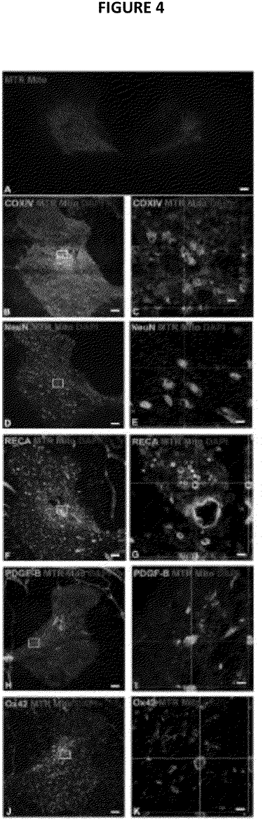

In the ongoing studies, mitochondria isolated from autologous or allogeneic rat soleus muscles will be labeled with MitoTracker Red CMX Ros dye (MTR Mito) and will be used to assess their distribution following transplantation. Intraspinal and/or subdural MTR Mito delivery both result in uptake by various cell types in spinal cords (FIGS. 3). Briefly, MTR Mito were either injected in the gray matter at four circumferential sites (25 µg/site, 100 µg total) at L1/L2 spinal levels [40] or they were delivered via subdural insertion of Gelfoam™ in which 100 µg MTR Mito were laden. After 48 hours, spinal cords were processed to show remarkably similar distribution of MTR Mito with both routes of administration (FIGS. 3 A,F ). Notably, injected MTR Mito were found juxtaposition to, but not taken up by neurons, and many of which appeared missing (FIGS. 3B,C ). In some other aspects, while subdural MTR Mito were also taken up by microglia/brain macrophages (FIGS. 3I,J), there was less evidence of proximal neuronal loss (FIGS. 3G,H ). Upon further evaluation of subdural delivery sites (FIGS. 4), spinal cord sections containing regions of MTR Mito dispersion were found to colocalize with endothelial cells, pericytes and microglia/macrophages, and intact neurons were found juxtaposition to MTR Mito, unlike intraspinal injections.

To enhance the uptake of mitochondria during MitoTxp, while minimizing injection induced secondary injury, it is possible to use an injectable dissolution-controlled delivery system. Thermogelling systems are an attractive solution as a means of prolonging retention at the intended site of action [42, 43], and while they have been used for cell delivery and tissue repair in SCI models [44-48], it is important to know what the transport potential of mitochondria is in the presence of polymers in the design of this system.

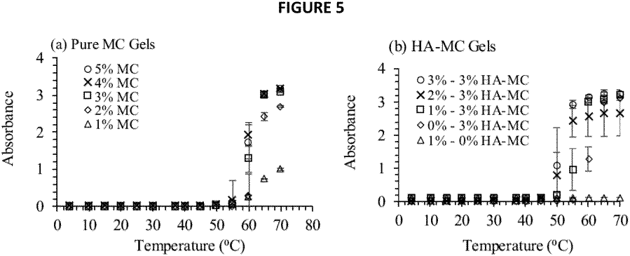

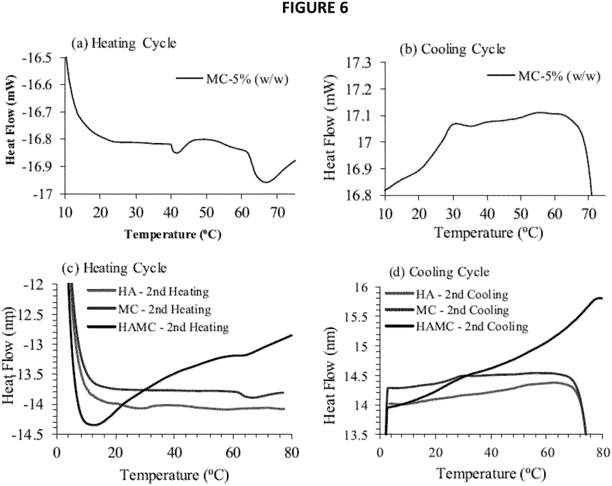

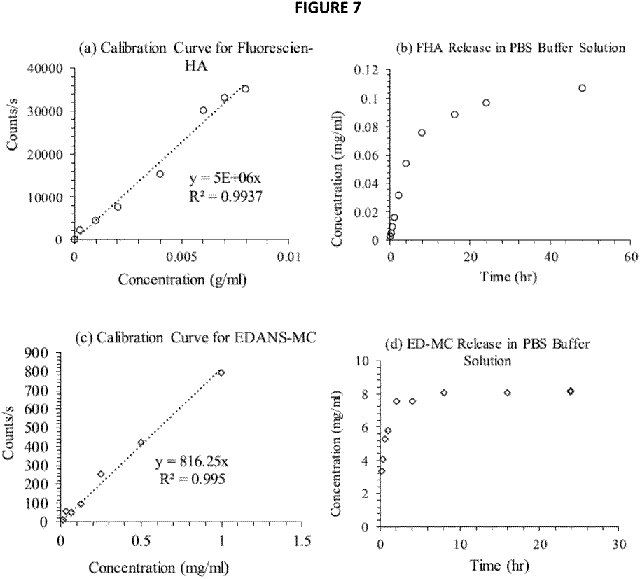

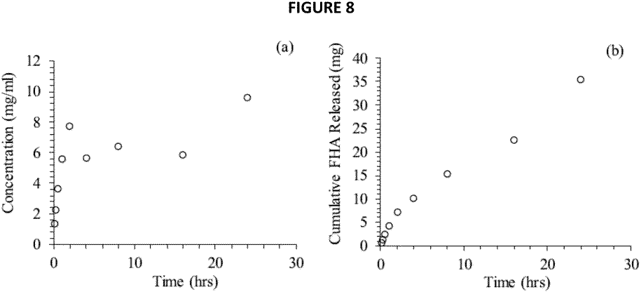

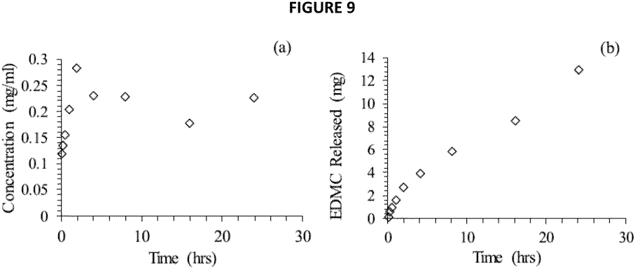

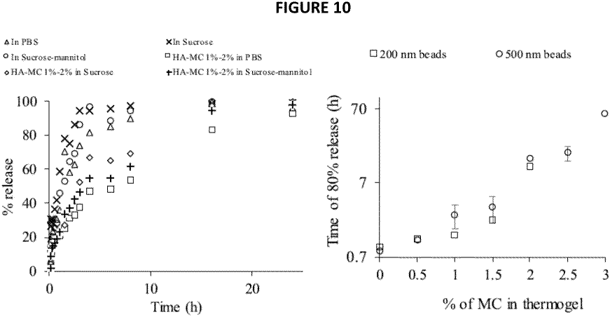

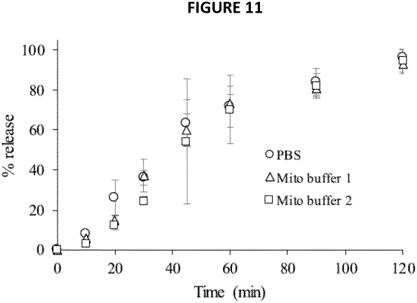

Preliminary diffusional studies in this regard using fluorescence correlation spectroscopy (FCS) of fluorescently labeled mitochondria have been performed [49]. Mobility of mitochondria tagged with Mitotracker Green FM in varying concentration (C=0-12% w/v) of polyethylene glycol (PEG, average MW 20 k) solution in isolation buffer (215 mM mannitol, 75 mM sucrose, 0.1% BSA, 20 mM HEPES, 1 mM EGTA and KOH-pH 7.2) with/without mitochondria substrate (5 mM pyruvate, 2.5 mM malate, 10 mM succinate) at 4° C. was measured by FCS, using a dual channel laser scanning confocal spectroscope couple with Nikon Ti-U microscope. FCS measures the fluorescence fluctuation in a control volume due to mobility of probe molecules and reports a correlation function (FIG. 5-inset), which shifts rightward with decreasing mobility, and was fitted to intensity fluctuation function due to Brownian motion to estimate the diffusivity of mitochondria (FIG. 5). Diffusivity (DD) is reduced with increasing polymer (and viscosity, µµ). Note that mitochondria are ~50% slower with substrate, due to their swelling in presence of substrate (radius, aa = 113 and 241 nm without/with substrate from Stokes-Einstein equation, D= k_{B}T/6πηR). Critically, it has been established that the bioenergetic integrity of isolated mitochondria is maintained at room temperature for 30 min in polymer solutions consisting of hyaluronate (HA; 1 wt%) and/or methylcellulose (MC; 2 wt%), as measured by OCR.

As seen in preliminary data, diffusion of mitochondria through polymer solutions is significantly diminished due to the increased tortuous length caused by the polymer chains. As this effect is only expected to increase in the case of a crosslinked hydrogel system, we plan to use an erodible, thermogelling polymer blend of HA, a naturally occurring well tolerated biopolymer, and MC, a common biopolymer used in multiple pharmaceutical and tissue engineering applications. Blends of HA (1 wt%) and MC (2-4 wt%) have recently been studied for their use as injectable drug delivery vehicles in ocular application [49]. Through tuning the polymer ratio, the erosion rate of the gel can be tuned from several hours to 7-10 days [51].

### Hypothetical 1: Transplantation of Isolated Mitochondria (Mitotxp) Can Be Delivered Through Diffusible Hydrogel Polymers

For this area for development, 12 adult female Sprague-Dawley (SD) rats (225-250 g) will be used for isolating mitochondria to establish best fabrication of a temperature-sensitive hydrogel that solidifies at body temperature that mitochondria can be suspended in to allow their controlled widespread diffusion into the underlying spinal cord parenchyma. The thermogel formulation will provide an ideal localized delivery vehicle for the stability of mitochondria during MitoTxp, with enhanced control over the aggregation, swelling and localized transport of mitochondria to reduce the technical limitation of standard injection delivery.

HA/MC blends will be synthesized and injected into PBS (pH 7.4) at 37° C. to evaluate thermogelling kinetics and dissolution properties. Variables to be studied to determine gelation and dissolution time include polymer blend ratios (0.1 to 10 HA/MC), total polymer solution concentration (0.1 to 3.0 wt%), and solution ionic strength. Thermogelling kinetics will be evaluated using a cone and plate viscometer. It is expected, that during thermogelling, some degree of mitochondrial release will occur prior to gelation. To asses this effect, additionally, fluorescently labeled latex beads (200 nm) will be used (as a mitochondrial analog) to determine the amount of loss observed prior to gelation. Afterwards, dissolution will be monitored via mass loss of formed gels and polymer release using dye modified HA and particle release. Once gels have been identified with a 1-hour dissolution window, mitochondria release will be assessed in vitro. As part of this work, the impact of thermogelling on mitochondrial suspension, size and aggregation will be evaluated using fluorescence correlation spectroscopy (FCS).

### Hypothetical 2: Subdural MitoTxp Will Significantly Maintain Bioenergetics After SCI

60 adult female Sprague-Dawley (SD) rats (225-250 g) will be divided into 6 groups (10/group): 1) Sham, 2) SCI + Vehicle, 3) SCI + Intraspinal MitoTxp 100 ug, 4) SCI + Subdural MitoTxp 50 ug, 5) SCI + Subdural MitoTxp 100 ug, and 6) SCI + Subdural MitoTxp 200 ug.

Following laminectomy of T12 vertebra, exposed spinal cords will be contused at the L1/L2 spinal level (250 kdyn) using Infinite Horizon impactor as we have reported [1, 10, 40]. Sham groups receive laminectomy only. The dosage of intraspinal MitoTxp is based on our published data [40] that will be compared with MitoTxp dose-response via polymeric subdural route. Injured rats will receive either intraspinal injections, as we have reported, or subdural delivery of healthy mitochondria isolated from soleus muscle and labeled with MitoTracker Red CMXRos versus vehicle 2-3 hours after injury. At 48 hours after injury, mitochondria from injured and sham spinal cords (1 cm segment centered on injury site) are isolated to assess bioenergetics in terms of oxygen consumption rate (OCR) using the Seahorse Bioscience XFe24 flux analyzer [40] that will be correlated with cellular ATP levels. Circulating free mitochondrial DNA could activate damage-associated molecular patterns (DAMPs) to induce immune responses and cell injury [56, 57], but recent studies from the pioneering McCully group have shown no significant inflammatory responses associated with systemic allogeneic MitoTxp [32, 58-60]. Mitochondrial associated DAMPs will be assessed by measuring cytochrome c release from mitochondria, levels of formyl peptides and mtDNA in circulation, and inflammatory cytokines IL1-β and TNF-α in injured tissue and blood.

For assessments of acute bioenergetics after combination therapies plus MitoTxp, a total of 160 rats will be divided into 8 groups (10/group for acute and 10/group for delayed MitoTxp): 1) Sham, 2) SCI + Vehicle, 3) SCI + MitoTxp, 4) SCI + ALC, 5) SCI + NACA, 6) SCI + ALC + MitoTxp, 7) SCI + NACA + MitoTxp, and 8) SCI + ALC + NACA + MitoTxp. Injury paradigm is as detailed in Aim 1.

Injured rats will receive injections of either vehicle or ALC (300 mg/kg, i.p.) [1] or NACA (150 mg/kg, i.p.) [10] or both at 3 hours post-injury followed by booster ALC and/or NACA injections at 6 hr. Injured rats in MitoTxp alone and combination groups will be transplanted with MTR Mito (dosage and delivery route determined in Aim 1) at either 3 hours post-injury (acute) or 24 hours post-injury (delayed). At 48 hours after injury, spinal cord tissues will be isolated and assessed for mitochondrial bioenergetics, ATP production, GSH levels and oxidative stress markers, as we’ve detailed [10, 61]. OCR results will guide Aim 3 for therapeutic time of administration.

For assessments of sub-acute neuroprotection/cellular incorporation after combination therapies plus MitoTxp, a total of 160 rats will be divided into 8 groups (10/group for acute and 10/group for delayed MitoTxp), as described in Experiment 2.1. We will investigate sub-acute time points since labeled exogenous mitochondria are dissipated within 1-week post-MitoTxp [40]. At 5 days post-injury, rats will be perfused and spinal cords processed for histological assessments of tissue sparing and mitochondrial incorporation into resident cells. Bioenergetic results from the treatment paradigms, both acute and delayed, will be compared to tissue sparing and cell-type incorporation propensities for correlations to address potential mechanisms. Stereological quantification is expected to show increased cellular incorporation and tissue sparing with combinatorial treatments.

### Hypothetical 3: Combinational Regimens Will Maximize Long-Term Functional Neuroprotection

120 rats (60 female, 60 male) will be divided into 5 treatment groups (12/group/sex): 1) SCI + Vehicle, 2) SCI + MitoTxp, 3) SCI + ALC + MitoTxp, 4) SCI + NACA + MitoTxp, and 5) SCI + ALC + NACA + MitoTxp.

Injured rats will receive ALC and/or NACA + MitoTxp (based on Aim 2). ALC will be administered daily for 3 weeks whereas NACA will be administered daily for 1 week. Animals will be assessed with BBB locomotor scale (weekly for 6 weeks) and terminal gait analysis for quantitative measures of hind limb function [10]. In parallel, Von Frey hair and heat hypersensitivity testing will be done [40], after which their spinal cords are processed for histology [10, 40, 62].

### Methods

Surgical procedures: All surgical procedures will follow the University of Kentucky’s Policy for Rodent Survival Surgery. Severe (250 kDyn) contusion SCI will be performed at L1/L2 spinal levels under aseptic conditions using sterilized instruments as we have previously reported [1, 10].

Isolation of muscle and spinal cord mitochondria: Naive Sprague-Dawley rats are asphyxiated with CO2 and decapitated and their soleus muscles are removed and placed in 2 mL of isolation buffer (215 mM mannitol, 75 mM sucrose, 0.1% bovine serum albumin [BSA], 20 mM HEPES, and pH adjusted to 7.2 with KOH) containing 1 mM EGTA and 0.025% trypsin. The muscles are chopped into smaller pieces and mechanically homogenize. A protease inhibitor is added to halt trypsin activity, followed by centrifugation at 1500 rcf, 4° C. for 5 minutes. The supernatant is removed and centrifuged at 13,000 rcf for 10 minutes at 4° C., after which the pellet is resuspended in isolation buffer and purified using Ficoll gradient (7.5%/10%) centrifugation at 32,000 rpm for 30 minutes, 4° C. as described previously [63]. The resulting final pellet containing purified mitochondria are labeled with MitoTracker Red CMXRos (MTR, Molecular Probes) as described by manufacturer and assessed for their respiratory capacity using Seahorse Flux Bio analyzer before transplantation into spinal cords, as described previously [40]. One-centimeter spinal cord segments are homogenized in isolation buffer and mitochondria are isolated using Ficoll gradient (7.5%/10%) centrifugation as described previously [1, 10, 40].

Transplantation of mitochondria: For intraspinal delivery, freshly isolated MTR labeled soleus muscle mitochondria will be microinjected into naive or injured spinal cords using a glass micropipette needle (World Precision Instruments, Sarasota, FL) pulled and beveled to a 20-30 µm inner diameter pore opening. Each injection consisted of 750 nL of either vehicle (isolation buffer with 5 mM pyruvate, 2.5 mM malate, and 10 mM succinate) or mitochondria suspended in vehicle; hence a total of 3 µL volume will be injected per spinal cord as we have reported [40]. For subdural delivery, for preliminary data, ~5mm incision was made in exposed L1 dura and 4 small pieces of sterilized Gelfoam™ impregnated with 100 ug of MTR Mito were inserted bilaterally under the dura that was closed using 8.0 dural sutures before covering with a thin membrane of sterilized agarose gel to seal the prep. For the hydrogel experiments, the prepared polymeric MitoTxp solutions will be injected intrathecally with modified Hamilton syringes with curved tips caudal to the injury.

Assessment of Mitochondrial Function: At designated terminal time points, mitochondrial function will be assessed in terms of 1) mitochondrial respiration using a Seahorse Bioscience extracellular flux analyzer as we have reported [10, 64] and 2) activities of mitochondrial enzymes complexes using Multidetection Microplate Reader (Bio-Tek Instruments, Winooski, VT) normalized to milligram of mitochondrial protein [41, 65].

Markers of mitochondrial oxidative damage: Measurements of oxidative damage markers, 3-nitrotyrosine or 4-hydroxynonenal, and protein carbonyls will be carried out by western blots as we have described [61].

Behavioral Assessments: Basso, Beattie, Bresnahan (BBB) Locomotor Rating Scale will be performed at 2-4 days post-injury and then weekly up to 6 weeks to test recovery of hindlimb function over time post-injury as described [66]. Rats will also be assessed for hindlimb pain hypersensitivity [40], as well as terminal gait analysis [10].

Tissue Processing for Histology and Immunohistochemistry: At designated terminal time points, rats will be overdosed with sodium pentobarbital and transcardially perfused with 0.1 M phosphate-buffered saline (PBS, pH 7.4) followed by 4% paraformaldehyde in PBS (for histology) and 30 mm segment of spinal cord is immediately dissected, post-fixed for several hours and serially cryosectioned at 20 µm for unbiased histological assessments, as we have reported [1, 10, 41]. Alternate tissue sections will be used for immunohistochemistry to estimate the propensities of cell-specific uptake of MTR mitochondria [40].

We claim:

1. A composition for protecting tissue at a site of injury, comprising a hydrogel with isolated mitochondria suspended therein.

2. The composition of claim 1, wherein the hydrogel comprises a homopolymer hydrogel, a copolymer hydrogel, or a multipolymer interpenetrating polymeric hydrogel.

3. The composition of claim 1, wherein the hydrogel comprises an amorphous or noncrystalline hydrogel, a semicrystalline hydrogel, or a crystalline hydrogel.

4. The composition of claim 1, wherein the hydrogel is responsive to temperature, electric field, magnetic field, light, pressure, sound, pH, ionic strength, sound, surrounding molecules and/or solvents.

5. The composition of claim 1, wherein the hydrogel is a thermo-gelling hydrogel.

6. The composition of claim 5, wherein the hydrogel is selected from the group consisting of hyaluronic acid (HA), methylcellulose (MC)N-isopropylacrylamide (PNiPAAm), chitosan, pluronic or poloxamers of poly(ethylene oxide)-b-poly(propylene oxide)-b-poly(ethylene oxide), polyethylene glycol (PEG), PEG acrylate, PEG thiol-ene, acrylic acid and esters thereof, methyl acrylate, butyl acrylate, ethyl acrylate, 2-ethylhexyl acrylate, gelatin, laminin, mucin, sericin, fibrin, alginate, heparin, and/or polyvinyl alcohol.

7. The composition of claim 5, wherein the hydrogel comprises hyaluronic acid (HA) and methylcellulose (MC).

8. The composition of claim 5, wherein thermo-gelling hydrogel may be of from about 0.1 to about 20% by weight of the composition.

9. The composition of claim 1, further comprising one or more supportive agents suspended therein, wherein the one more supportive agents provide for mitochondrial health and/or improve tissue healing and are selected from the group consisting of water, buffers, polymers, chelating agents, surfactants, emulsifiers, vitamin C, vitamin D, vitamin E, vitamin B6, vitamin B12, amino acids, branched amino acids, iron, magnesium, calcium, sodium, potassium, silver, gold, manganese, phosphates, sulfates, chlorides, fructose, NADH, NADPH, an anti-inflammatory, an anti-fibrotic, a steroid, an anti-bacterial, an anti-viral, an anti-fungal, collagen, albumin, or any combination thereof.

10. The composition of claim 1, further comprising N-acetylcysteine amide (NACA).

11. The composition of claim 10, wherein NACA is at a weight percentage of from about 0.1 to about 20% by weight of the thermo-gelling hydrogel.

12. The composition of claim 1, further comprising acetyl-L-carnitine (ALC).

13. The composition of claim 12, wherein ALC is at a weight percentage of from about 0.1 to about 20% by weight of the thermo-gelling hydrogel.

14. The composition of claim 1, further comprising ALC and NACA.

15. The composition of claim 1, further comprising a nano and/or microparticle that provides controlled release of one or more supportive agents therein.

16. The composition of claim 15, wherein the nano and/or microparticles are comprised of lipids, biodegradable polymers, biodurable polymers, saccharides, and/or carbohydrates.

17. The composition of claim 16, wherein the nano and/or microparticles are comprised poly(ethylene glycol) (PEG), poly(lactic acid) (PLA), poly glycolic acid (PGA), poly(lactic-coglycolic acid) (PLGA), poly(caprolactone) (PCL), poly (β-amino ester) (PβAE), chitosan, starch, cholesterol, distearoyl phosphocholine, 1,2-dioleoyl-3-trimethylammoniumpropane (DOTAP), dioleoylphosphatidylethanolamine (DOPE), lipofectamine, dioleoylphosphatidylcholine (DOPC), phosphatidylethanolamine (dioleoyl PE), 3β-[N-(N′,N′-dimethylaminoethane)-carbamoyl]-cholesterol (DC-Chol), or a combination thereof.

18. The composition of claim 1, wherein mitochondria comprises about 0.1 to about 30% by weight of the composition.

19. A method for treating a site of injury in a subject comprising administering the composition of claim 1 directly or proximal to a site of injury.

20. The method of claim 19, wherein the hydrogel comprises a thermo-gelling hydrogel that transforms from sol to gel at a subject’s body temperature.
